(19) United States
(12) Patent Application Publication
(10) Pub. No.: US 2005/0154263 A1
(43) Pub. Date: Jul. 14, 2005

(54) Device for spreading and holding open a body cavity

(76) Inventor: Nady E. Nady, Skillman, NJ (US)

Correspondence Address: Gregory C. Houghton, Esq.;Mathews, Collins, Shepherd & McKay, P.A., 100 Thanet Circle, Suite 306, Princeton, NJ, 08540, US

(21) Appl. No.: 10/756,525

(22) Filed: Jan. 13, 2004

(51) Int. Cl. A61B 1/32

(52) U.S. Cl. 600/210

(57) ABSTRACT

A device useful for spreading apart and holding open a body cavity which includes an illumination device for illuminating the body cavity and an irrigation channel for irrigating the body cavity during diagnostic and/or surgical procedures.

### BACKGROUND OF THE INVENTION

1. Field of the Invention

The present invention relates to a device for spreading apart and holding open a body cavity which includes an illumination device for illuminating the body cavity and an irrigation channel for irrigating the body cavity during diagnostic and/or surgical procedures

2. Related Art

Certain surgical and diagnostic devices are known for examining vaginal and other body cavities. For example, devices for retracting the vaginal canal for gynecological procedures include medical or surgical instruments known variously as retractors or speculums.

These devices retract the vaginal side walls which normally obstruct the doctor's view into the patient's cervix. During a surgical procedure, the surgeon needs to insert a clamp, a source of lighting, a suctioning catheter, and irrigation. This further obstructs the surgeon's view of the cervix which might interfere with the surgical procedure being performed.

Also, conventionally, separate upper, side and lower manual retractors are employed for vaginal procedures. Because these conventional retractors must be held in place manually, the assistants holding these retractors crowd the procedure area which contributes to the difficulty of the procedure being performed by the surgeon.

It is therefore desirable to provide a device to spread and hold open a body cavity that includes an illumination device and an irrigation channel to assist a surgeon in performing diagnostic or surgical procedures.

### SUMMARY OF THE INVENTION

The present invention provides a device for spreading apart a body cavity during diagnostic or surgical procedures that includes pivotally connected cross arms having forward end portions with plurality of blades and rearward end portions with hand grips. The connection between the cross arms permits pivotal movement between the cross arms and permits a predetermined longitudinal relative shifting of said cross arms, wherein said plurality of blades are adapted to spread apart the body cavity during diagnostic or surgical procedures.

The device includes an illumination device. The illumination device is attached to the pivotally connected cross arms and provides illumination to the body cavity during diagnostic or surgical procedures. The device also includes an irrigation channel attached to the pivotally connected cross arms, wherein the irrigation channel provides irrigation to the body cavity during diagnostic or surgical procedures.

Also, the device can be locked with a locking device connected to the cross arms wherein the locking device holds the forward end portion of the cross arms in an open position.

### BRIEF DESCRIPTION OF THE DRAWINGS

The advantage, nature, and various additional features of the invention will appear more fully upon consideration of the illustrative embodiments now to be described in detail in connection with accompanying drawing wherein:

FIG. 1 is a schematic diagram of a device for spreading apart and holding open a body cavity which includes an illumination device for illuminating the body cavity and an irrigation channel for irrigating the body cavity during diagnostic and/or surgical procedures according to an exemplary embodiment of the invention.

### DETAILED DESCRIPTION OF THE INVENTION

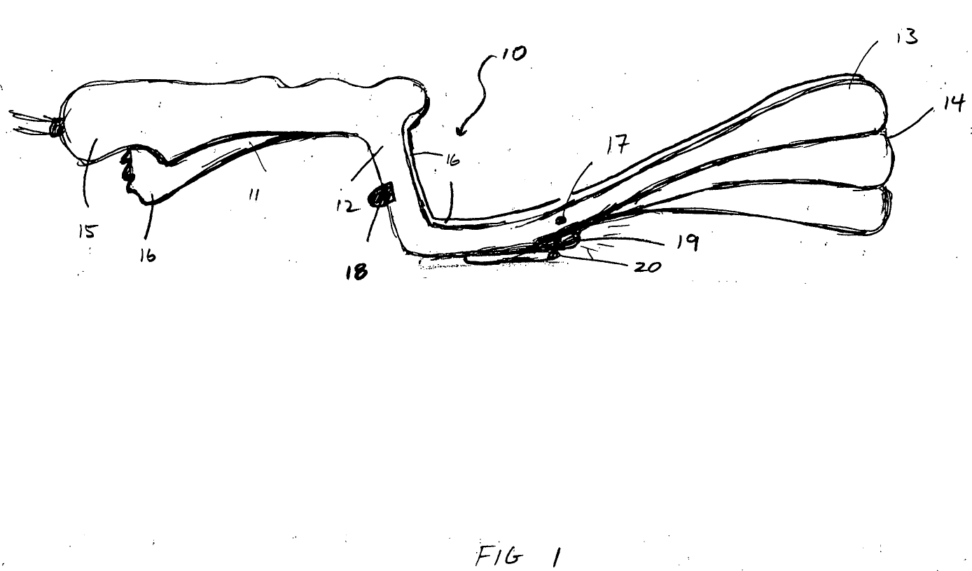

FIG. 1 shows device 10 in accordance with the invention. Device 10 spreads open and holds open a body cavity and provides an illumination device for illuminating the body cavity during diagnostic and/or surgical procedures and an irrigation channel for irrigating the body cavity during diagnostic and/or surgical procedures. Device 10 thereby decreases the need for secondary illumination devices and irrigation sources and the need for surgical assistants who are used to manually hold open the body cavity with conventional retractors.

Device 10 includes cross arms 11 and 12 which are of elongate construction. Cross arms 11 and 12 have a plurality of blades 13 and 14 at the front end portion and hand-grips 15 and 16 at the rear end portion.

Cross arms 11 and 12 are pivotally connected to each other with connection 17. The closing of hand-grips 15 and 16 (i.e. bringing hand-grip 15 toward hand-grip 16) enables plurality of blades 13 and 14 to be moved away from each other into an open position.

Connection 17 can be a pivot point for connecting cross arms 11 and 12 and is well understood by those skilled in the art. For example, instruments that have pivotally connected cross arms include scissors, pliers, and hemostats.

Cross arms 11 and 12 can be locked to one another by locking device 18. Locking device 18 holds front end portion of cross arms 11 and 12 in an open position incrementally over an angular range of pivotal movement of cross arms 11 and 12. One exemplary embodiment of locking device 18 is a locking latch system used in hemostats. Other locking devices which are known to those skilled in the art are equally suitable which include but are not limited to a latch-type system used by pliers and/or clippers.

Device 10 includes illumination device 19. Illumination device 19 is attached to either cross arm 11 or cross arm 12 or both cross arms 11 and 12 and enables a surgeon or physician to illuminate a body cavity during diagnostic and/or surgical procedures. Device 10 includes irrigation channel 20. Irrigation channel 20 is attached to either cross arm 11 or cross arm 12 or both cross arms 11 and 12 and enables a surgeon or physician to irrigate the body cavity during diagnostic and/or surgical procedures. Irrigation channel 20 can include a suctioning catheter to assist in suctioning and removing fluids during diagnostic and/or surgical procedures. In a further embodiment illumination device 19 and irrigation channel 20 can be removably attached to either cross arm 11 or cross arm 12 or both cross arms 11 and 12.

Illumination devices are well understood by those skilled in the art and include but are not limited to any illumination device understood by those skilled in the art that can be adapted to be used with the device of the present invention. These include the current illumination sources that are employed to illuminate surgical or diagnostic areas.

Irrigation channels are well understood by those skilled in the art and include but are not limited to any irrigation source or device understood by those skilled in the art that can be adapted to be used with the present invention. For example, a hose or tube for providing water for irrigation.

Suctioning catheters are well understood by those skilled in the art and include but are not limited to any suctioning source or device understood by those skilled in the art that can be adapted to be used with the present invention.

Device 10 can be formed of metal or plastic. Device 10 can be reusable, replaceable and disposable.

Plurality of blades 13 and 14 have at least two flexible blades (i.e. at least one flexible blade per cross arm) which can be formed of metal or an elastomeric material. For example, an elastomeric material can be polyurethane, poly(vinyl chloride) or any other suitable material. The flexibility of the blade allows the blade to conform to the interior walls of the body cavity. In another embodiment of the present invention the plurality of blades can be attached to an elastomeric membrane wherein the elastomeric membrane stretches out, similar to the webbing of a duck's foot or a flipper, and attaches to another blade of the plurality of blades. The elastomeric membrane can be formed of polyurethane, poly(vinyl chloride) or any other suitable material.

The term body cavity includes but is not limited to any body orifice, such as a vaginal canal, anus, and mouth, etc. Further, a body cavity also includes surgical incisions made in a body which include but are not limited to incisions made in the abdomen.

The term fluids includes but is not limited to any fluid that may be present in a body cavity during diagnostic and/or surgical procedures, such as water, and blood, etc.

It is to be understood that the above-described embodiments are illustrative of only a few of the many possible specific embodiments which can represent applications of the principles of the invention. Numerous and varied other arrangements can be readily devised in accordance with these principles by those skilled in the art without departing from the spirit and scope of the invention.

1. A device for spreading apart a body cavity during diagnostic or surgical procedures comprising:
pivotally connected cross arms having forward end portions with plurality of blades and rearward end portions with hand grips, the connection between said cross arms permitting pivotal movement and enabling said plurality of blades to be laterally moved away from each other in the same plane into an open position;
a locking device connected to said cross arms, said locking device holds said cross arms in said open position incrementally over an angular range of pivotal movement of said cross arms, wherein said plurality of blades are adapted to spread apart the body cavity during diagnostic or surgical procedures; and
an illumination device, said illumination device attached to said pivotally connected cross arms, wherein said illumination device provides illumination to the body cavity during diagnostic or surgical procedures.

2. The device according to claim 1 further comprising an irrigation channel, said irrigation channel attached to said pivotally connected cross arms, wherein said irrigation channel provides irrigation to the body cavity during diagnostic or surgical procedures.

3. The device according to claim 2 wherein said irrigation channel comprises a suction catheter, said suction catheter removes fluids from the body cavity during diagnostic or surgical procedures.

4. The device according to claim 1 wherein said illumination device is removably attached to said pivotally connected cross arms.

5. The device according to claim 2 wherein said irrigation channel is removably attached to said pivotally connected cross arms.

6. (canceled)

7. (canceled)

8. The device according to claim 1 wherein said plurality of blades comprise at least two flexible blades.

9. The device according to claim 8 wherein said plurality of blades are connected to one another by an elastomeric membrane.

10. The device according to claim 8 wherein said flexible blades are made from an elastomeric material.

11. The device according to claim 1 wherein said device is reusable.

12. A device for spreading apart a vaginal canal during diagnostic or surgical procedures comprising:
pivotally connected cross arms having forward end portions with plurality of blades and rearward end portions with hand grips, the connection between said cross arms permitting pivotal movement and enabling said plurality of blades to be laterally moved away from each other in the same plane into an open position;
a locking device connected to said cross arms said locking device holds said cross arms in said open position incrementally over an angular range of pivotal movement of said cross arms, wherein said plurality of blades are adapted to spread apart the vaginal canal during diagnostic or surgical procedures; and
an illumination device, said illumination device attached to said pivotally connected cross arms, wherein said illumination device provides illumination to the vaginal canal during diagnostic or surgical procedures.

13. The device according to claim 12 further comprising an irrigation channel, said irrigation channel attached to said pivotally connected cross arms, wherein said irrigation channel provides irrigation to the vaginal canal during diagnostic or surgical procedures.

14. The device according to claim 13 wherein said irrigation channel comprises a suction catheter, said suction catheter removes fluids from the vaginal canal during diagnostic or surgical procedures.

15. The device according to claim 12 wherein said illumination device is removably attached to said pivotally connected cross arms.

16. The device according to claim 13 wherein said irrigation channel is removably attached to said pivotally connected cross arms.

17. (canceled)

18. (canceled)

19. The device according to claim 12 wherein said plurality of blades comprise at least two flexible blades.

20. The device according to claim 12 wherein said plurality of blades are connected to one another by an elastomeric membrane.

21. The device according to claim 19 wherein said flexible blades are made from an elastomeric material.

22. The device according to claim 12 wherein said device is reusable.

23. A method for spreading and holding open a vaginal canal while providing illumination and irrigation to the vaginal canal during diagnostic or surgical procedures, the method comprising the steps of:
inserting a device comprising pivotally connected cross arms having forward end portions with plurality of blades and rearward end portions with hand grips, the connection between said cross arms permitting pivotal movement and enabling said plurality of blades to be laterally moved away from each other in the same plane into an open position;
a locking device connected to said cross arms said locking device holds said cross arms in said open position incrementally over an angular range of pivotal movement of said cross arms; an illumination device, said illumination device attached to said pivotally connected cross arms; and an irrigation channel, said irrigation channel attached to said pivotally connected cross arms;
moving said plurality of blades away from each other spreading the vaginal canal open; and
illuminating the vaginal canal with said illumination device during the diagnostic or surgical procedures.

24. The method according to claim 23 further comprising the step of irrigating the vaginal canal with said irrigation channel during the diagnostic or surgical procedures.

25. (canceled)

26. (canceled)
